SEQUENCE LISTING (8 sequences)

SEQ ID NO: 1 - protein, 332 aa, Geobacillus stearothermophilus 10
MAKTVETVLGPVPVEQLGKTLIHEHFLFGYPGFQGDVTRGTFREDEALRVAVEAAEKMKRHGIQTVVDPTPNDCGRNPAFLRRVAEETGLNIICATGYYYEGEGAPPYFQFRRLLGTAEDDIYDMFMAELTEGIADTGIKAGVIKLASSKGRITEYEKMFFRAAARAQKETGAVIITHTQEGTMGPEQAAYLLEHGADPKKIVIGHMCGNTDPDYHRKTLAYGVYIAFDRFGIQGMVGAPTDEERVRTLLALLRDGYEKQIMLSHDTVNVWLGRPFTLPEPFAEMMKNWHVEHLFVNIIPALKNEGIRDEVLEQMFIGNPAALFSAHHHHHH

SEQ ID NO: 2 - DNA, 981 bp, Geobacillus stearothermophilus 10
atggctaaaactgtcgaaaccgtccttggcccagtgccggtggaacagcttggcaaaacgctcatccacgagcatttcctcttcggctatccagggtttcaaggcgatgtgacgcgcggcacgttccgcgaagacgaggcgcttcgcgtcgcagtcgaggcggcggaaaagatgaagcggcacggcattcaaacggttgtcgatcccacgccgaacgactgcgggcgcaacccggcatttttgcggcgcgttgctgaagagacgggactgaacattatttgcgccaccggctattattatgaaggggaaggggcgccgccgtacttccaattccgccggcttctcggaacagcggaagatgatatttacgacatgtttatggccgagctgaccgagggcattgccgataccggaatcaaggcgggtgtcatcaagctcgcctcgagcaaagggcgcatcaccgagtacgaaaagatgttcttccgcgctgccgcccgcgcgcaaaaagagacgggtgcggtcatcatcacccatacgcaagaaggaacgatggggccggaacaagccgcctatttgcttgagcacggcgccgatccgaaaaaaattgtcatcggccatatgtgcggcaacacggacccggactatcatcgaaagacgcttgcttacggcgtttacattgcgtttgaccgcttcggcatccaagggatggtcggcgcgccgactgatgaggagcgggtgcggacgctccttgctctgctccgcgatgggtacgagaaacaaattatgctgtcgcatgacactgtcaacgtttggctcggtcgtccgtttacgctgccggaaccgtttgcggaaatgatgaaaaattggcatgtcgagcatttgtttgtgaacatcatccccgcgctgaaaaatgaaggaatccgcgacgaagtgcttgagcaaatgttcatcggcaatccggcggcgctgttctcggcttga

SEQ ID NO: 3 - protein, 334 aa, Geobacillus stearothermophilus F
MKKGMVETVCGPVPASELGKTLIHEHFVFGYPGFQGDVTLGPLRFEEALEAGIAVAQKMMAHGIQTVVDPTPNDCGRNPELLRRISEATGLNIICATGYYYEGEGATPYFKFRRLLGTAEEEIYDMFMAEITNGIAGTGIKPGVIKLASSKNRITEYEKMFFRAAARVQKETGIVIITHTQEGTMGPEQAAYLLEHGAVPQKIVIGHMCGNTDPEYHKKTLQYGVYIAFDRFGLQGMVGAPTDEERIRTLLVLLREGFADKIMLAHDTVNIWLGRPLQLPEPFATMTKNWHVEHVFEHIIPILTKEGVTEEQLEQMFLKNPAALFASGHHHHHH

SEQ ID NO: 4 - DNA, 978 bp, Geobacillus stearothermophilus F
atgaaaaaaggaatggtggagactgtctgcggccctgtgccggcgagcgagcttggcaaaacgctgatccatgagcattttgtctttgggtatcccgggtttcaaggcgatgtgacgctcggaccgcttcgctttgaggaagcgctcgaggcgggaattgccgtcgcgcaaaaaatgatggcccatggcatccaaaccgttgtcgatccgacgccgaatgactgcggccgcaatccggagctgctccgccgcatttccgaagcgaccgggctgaacattatctgtgcaactggatattattacgaaggcgaaggggcaacgccgtattttaagtttcgccggctgctcggtacggcggaagaagaaatatatgacatgtttatggcggaaattacaaacggcattgccggcaccggcattaaaccaggcgtcatcaaactggcgtcaagcaaaaaccggatcaccgaatatgaaaaaatgtttttccgcgccgcggcccgcgtgcaaaaagaaacaggaatcgtgattatcacccatacgcaggaaggaacgatggggccggaacaggcggcatacttgctcgagcacggtgccgttccgcaaaagattgtcatcggccatatgtgcggaaacaccgatccggaataccataaaaagacgctgcaatacggagtgtacatcgcttttgaccgctttggccttcaaggtatggtcggtgccccgacggatgaagaacggatccgcacgttgctcgtgctgcttcgcgaaggctttgctgataagattatgctcgctcatgataccgtcaatatttggctcggccgtccgctgcagctgccagagccatttgcgacgatgacgaaaaactggcatgtcgaacatgtatttgaacatattattccaattttaacgaaagaaggagtgacagaggaacagctagagcaaatgtttttgaaaaatcctgccgctttgtttgcg

SEQ ID NO: 5 - protein, 314 aa, Sulfolobus solfataricus
MRIPLVGKDSIESKDIGFTLIHEHLRVFSEAVRQQWPHLYNEDEEFRNAVNEVKRAMQFGVKTIVDPTVMGLGRDIRFMEKVVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKPKLAPRWSITLIFEDTIPFLKRNGVNEEVIATIFKENPKKFFS

SEQ ID NO: 6 - DNA, 945 bp, Sulfolobus solfataricus
atgagaataccattagttgggaaagattcaatagaatctaaggacataggatttacgctaattcatgaacatttaagagtttttagcgaagcggtcagacaacaatggccccatctatataacgaagatgaggagttcagaaacgctgtaaatgaggttaaaagggcaatgcaatttggagtaaagactatagtagatcccactgtaatgggattgggtagggacatcagatttatggaaaaagtggttaaggctaccgggataaatttagttgcggggacggggatttacatatatatcgacttacctttctatttcttaaataggtcaattgatgagatagctgacttgtttattcatgatataaaagagggaatacaaggtactctcaataaagctggcttcgtaaagatagctgcagatgaacctgggatcacaaaggatgtggagaaggtaataagggctgctgccatagcaaacaaagagactaaagtaccaataattacccactctaacgctcacaataacaccggattagaacagcaaagaatattgactgaagaaggtgttgatccagggaaaatattaataggtcatttaggtgatacagataatatagattacataaagaagatagcagataagggatcctttattggattagatagatatggtttagatttattcctacctgttgataagagaaatgaaacgaccttaagactaatcaaagatggttattcagataagataatgatctctcacgattattgttgcacaatcgactggggaactgcaaaaccagaatataaacctaagcttgctccaagatggagtataactctaatatttgaggatacgataccgttcttaaagagaaatggagtgaatgaagaggttatagctacaatatttaaggaaaatccgaaaaagttcttcagctaa

SEQ ID NO: 7 - protein, 314 aa, Sulfolobus solfataricus
MRIPLVGKDSIESKDIGFTLIHEHLRVFSEAVRQQWPHLYNEDEEFRNAVNEVKRAMQFGVKTIVDPTVMGLGRDIRFMEKVVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDIGTAKPEYKPKLAPRWSITLIFEDTIPFLKRNGVNEEVIATIFKENPKKFFS

SEQ ID NO: 8 - DNA, 945 bp, Sulfolobus solfataricus
atgagaataccattagttgggaaagattcaatagaatctaaggacataggatttacgctaattcatgaacatttaagagtttttagcgaagcggtcagacaacaatggccccatctatataacgaagatgaggagttcagaaacgctgtaaatgaggttaaaagggcaatgcaatttggagtaaagactatagtagatcccactgtaatgggattgggtagggacatcagatttatggaaaaagtggttaaggctaccgggataaatttagttgcggggacggggatttacatatatatcgacttacctttctatttcttaaataggtcaattgatgagatagctgacttgtttattcatgatataaaagagggaatacaaggtactctcaataaagctggcttcgtaaagatagctgcagatgaacctgggatcacaaaggatgtggagaaggtaataagggctgctgccatagcaaacaaagagactaaagtaccaataattacccactctaacgctcacaataacaccggattagaacagcaaagaatattgactgaagaaggtgttgatccagggaaaatattaataggtcatttaggtgatacagataatatagattacataaagaagatagcagataagggatcctttattggattagatagatatggtttagatttattcctacctgttgataagagaaatgaaacgaccttaagactaatcaaagaattttattcagataagataatgatctctcacgattattgttgcacaatcgactggggaactgcaaaaccagaatataaacctaagcttgctccaagatggagtataactctaatatttgaggatacgataccgttcttaaagagaaatggagtgaatgaagaggttatagctacaatatttaaggaaaatccgaaaaagttcttcagctaa

(19) United States
(12) Patent Application Publication
(10) Pub. No.: US 2024/0101996 A1
(43) Pub. Date: Mar. 28, 2024

(54) Enzymes Entrapped in Organopolysiloxane Matrix

(71) Applicant: JL Biosciences, Inc., La Grange, IL (US)

(72) Inventors: John Aikens, La Grange Park, IL (US); LeAnne M. Cabalka Tourtelotte, Valley Village, CA (US)

(21) Appl. No.: 18/038,077

(22) PCT Filed: Nov. 23, 2021
(86) PCT No.: PCT/US2021/060583

§ 371 Date: May 22, 2023

Related U.S. Application Data
(60) Provisional application No. 63/117,648, filed on Nov. 24, 2020.

(51) Int. Cl. C12N 9/96; A61K 38/46; A61K 47/34; C12N 9/18

(52) U.S. Cl. CPC C12N 9/96; A61K 38/465; A61K 47/34; C12N 9/18; C12Y 301/01081

(57) ABSTRACT

A stabilized enzyme product comprising composite particles that comprise an enzyme component within a nanoporous organopolysiloxane matrix. The enzyme component is capable of hydrolyzing one or more acyl-homoserine lactones and constitutes about 0.01 wt % to about 40 wt % of the composite particles on a dry weight basis. The composite particles have a density in a range of about 0.02 g/cm³ to about 0.5 g/cm³, an accessible surface area in a range of about 250 m²/g to about 600 m²/g, and fractal three-dimensional structures.

### CROSS-REFERENCE TO RELATED APPLICATIONS

This patent application claims priority to pending U.S. Provisional Patent Application Ser. No. 63/117,648, filed Nov. 24, 2020, and entitled, “Enzymes Entrapped in Organopolysiloxane Matrix,” the entire disclosure of which is incorporated herein by reference.

### STATEMENT REGARDING FEDERALLY SPONSORED RESEARCH OR DEVELOPMENT

Not applicable.

### FIELD OF THE INVENTION

Compositions comprising bioactive enzymes entrapped in organopolysiloxane matrix and methods for treating intestinal diseases, and methods for entrapping the enzymes in the organopolysiloxane matrix.

### BACKGROUND OF INVENTION

Orally delivered enzymes have a potential as therapeutics for treating gut lumen diseases, including inflammatory intestinal diseases such as inflammatory bowel disease (IBD), ulcerative colitis and Crohn's disease. Orally delivered enzymes may also help patients with pancreatic insufficiency. Bioactive enzymes may be also used for silencing the microbiome quorum signaling which is believed to play an important role in community related microbial behaviors, including biofilm formation and maintenance, virulence and microbial swarming in the gut lumen. In Gram negative bacteria, quorum signals are typically carried by acylhomoserine lactones. In Gram positive bacteria, quorum signals are typically carried by small post translationally modified secreted peptides.

Certain enzymes, including, but not limited to, acylhomoserine lactone hydrolases and organophosphate hydrolases are effective at hydrolyzing acylhomoserine lactone molecules. In vitro experiments in model systems have shown that quorum responsive microbial processes such as biofilm formation in Pseudomonas aeruginosa and pigment formation in Chromobacterium violaciens can be inhibited when cultures of these Gram negative bacteria are treated with enzymes capable of hydrolyzing acylhomoserine lactones.

In the case of Gram positive organisms within the microbiome, post-translationally modified peptides from the Corn C gene are secreted and act to carry quorum signals between Gram positive organisms. While highly diverse among strains of Gram positive bacteria, the Corn C peptide possess a consensus sequence (EXXA/GE) in the mature secreted peptide which can be recognized by trypsin-like proteases. Proteolytic degradation of the Corn C peptide results in the silencing of Gram positive microbe dependent quorum signals. Quorum signals derived from Gram positive or Gram negative organisms can also influence other microorganisms. Therefore, Gram positive organisms can respond to Gram negative quorum related responses and vice versa.

Certain enzymes, including trypsin-like proteases, acylhomoserine lactone hydrolases and organophosphate hydrolases, may find an application in controlling signaling of Gram negative and Gram positive bacteria in gut.

However, it is difficult, if not impossible, to orally deliver bioactive enzymes to the lower gut of a patient because proteins, including enzymes, are efficiently denatured and degraded in the upper gut.

In order to stabilize an enzyme, enzyme immobilization to a certain support can be used. A number of materials and techniques have been developed for enzyme immobilization, including synthetic polymer resins, natural polymers, and natural inorganic and synthetic minerals.

Overall, three enzyme immobilization processes are known: 1) physical adsorption to surfaces through electrostatic interaction, 2) chemical crosslinking to surfaces by reactive groups fixed to the surface, and 3) physical trapping of enzymes in matrices. In most cases regardless of the method employed, enzymes are loaded onto a support. Generally, the enzyme loading capacity of the support is limited by available surface area and packing density within the chosen material and is often expressed as milliequivalents of protein per support.

Immobilizing enzymes provides a number of advantages and enables a performance in enzyme-mediated processes, including resistance to denaturation, improved handling and ease of downstream processing. Despite these advantages, each immobilization support and method has limitations.

Chemisorbed enzymes are vulnerable to leaching as binding interactions are equilibrium dependent. As such, the solution environment needs to be carefully monitored to avoid enzyme wash off.

Covalent cross-linking provides a way to fix proteins permanently to the support through chemical reactions between support functional groups and specific amino acid residues found on the enzyme. The consequent covalent linkages insure that enzymes remain fixed to the surfaces. However, this approach leads to low loading densities, high costs associated with substrates and potential interference of the crosslink site with enzyme active sites. One specific unique example of crosslinking enzymes involves chemistry designed to stabilize enzyme crystals. In this case the support material is the crystalline enzyme itself which provides a theoretical upper limit for enzyme loading in a solid state.

Since at least the 1990s, enzyme entrapment in solid matrices has been used to stabilize enzyme activities and create materials that can be readily handled and recovered from solutions. The most common technique involves the entrapment of enzymes within a matrix that is assembled in situ. Polysaccharides such as alginate can be dispersed and solubilized in aqueous media that also contains co-solubilized enzymes. Calcium salts are then introduced which ligate to the alginate creating a solid matrix. While effective, alginate-based immobilization matrices are very soft and shear sensitive. In addition, these materials cannot be dehydrated and rehydrated.

As an alternative, siloxane monomers, particularly tetramethoxysilane and tetraethoxysilane, have been applied to enzyme entrapment. However, the historical limitation to siloxane-based immobilization has been that the monomers are poorly water-soluble and once they are hydrolyzed, they introduce alcohol solvents which can denature the enzymes to be entrapped. The process also calls for high shear conditions such as sonication which is needed to emulsify the siloxane monomers prior to polymerization via solution gel chemistry.

The general understanding prior to this disclosure is that siloxane-based enzyme immobilization is a difficult and costly process which cannot be scaled efficiently for commercial applications. In addition, the matrix is generally a pure silicate which has variable surface chemistry affecting morphology, pore size and chemical compatibility to enable dynamic equilibrium with the environment.

### SUMMARY OF INVENTION

In one embodiment, the invention is directed to a method of stabilizing an enzyme. The method comprising: forming a hydrolysis reaction mixture that comprises a siloxane monomer component, an acid component, and a solvent component, wherein:
the acid component is at an amount sufficient for the hydrolysis reaction mixture to have a pH in a range of about 1 to about 6 and to catalyze the hydrolysis of the siloxane component thereby producing an oligo-siloxane sol that comprises siloxane oligomers;
the solvent component comprises water at an amount such that a mol ratio of water to silicon of the siloxane monomer component is in a range of about 0.2:1 to about 100:1; and
the siloxane monomer component comprises:
   one or more tetraalkoxysilane monomers having the general formula Si(OR)₄ or (OR)₃Si(OSi(OR)₂)ₙOSi(OR)₃; and
   one or more organo-alkoxysiloxane monomers having the general formula Si(OR)₄₋ₓR′ₓ;
wherein:
   n is in the range of 0-100;
   x is in the range of 1 to 3;
   each of the R groups is selected from the group consisting of methyl, ethyl, propyl, isopropyl, and butyl alkyl; and
   each R′ is selected from the group consisting of methyl, ethyl, propyl, isopropyl, butyl, octyl, hexadecyl, octadecyl, phenyl, trimethylaminopropyl, aminopropyl, aminobutyl, methylphosphonyl, hydroxymethyl, hydroxypropyl, methoxypropyl, methoxymethyl, acetoxymethyl, acetoxypropyl, acetoxypolyethlyeneoxypropyl, benzyl, carboxymethoxyethyl, epoxyhexyl, glycidoxypropyl, isocyanopropyl, and phenethyl; and
wherein the one or more organo-alkoxysiloxane monomers are at an amount that is greater than 0 mol % and no more than about 30 mol % of the siloxane monomer component on a silicon mol basis.

The method further comprises:
removing any non-aqueous solvents from the oligo-siloxane sol to form an aqueous oligo-siloxane sol;
forming a gelation reaction mixture that comprises the aqueous oligo-siloxane sol, an enzyme component, and a base component and/or a buffer component, wherein the gelation reaction mixture has a pH in a range of about 7 to about 10, wherein base component and/or the buffer component catalyze the gelation of the aqueous oligosiloxane sol thereby producing a gel that comprises a solid phase and liquid phase, wherein the enzyme component is capable of hydrolyzing one or more acyl-homoserine lactones, wherein the solid phase comprises the enzyme component entrapped in nanoporous organopolysiloxane, and wherein the entrapped enzyme component constitutes about 0.01 wt % to about 40 wt % of the solid phase on a dry weight basis; and
curing the gel to form a solid hydrogel;
   thereby stabilizing the enzyme.

In another embodiment, the present invention is directed to a stabilized enzyme product comprising composite particles that comprise an enzyme component within a nanoporous organopolysiloxane matrix wherein:
the enzyme component is capable of hydrolyzing one or more acyl-homoserine lactones;
the enzyme component constitutes about 0.01 wt % to about 40 wt % of the composite particles on a dry weight basis;
the composite particles have a density in a range of about 0.02 g/cm³ to about 0.5 g/cm³, an accessible surface area in a range of about 250 m²/g to about 600 m²/g, and fractal three-dimensional structures; and the nanoporous organopolysiloxane is derived from a siloxane monomer component that comprises:
one or more tetraalkoxysilane monomers having the general formula Si(OR)₄ or (OR)₃Si(OSi(OR)₂)ₙOSi(OR)₃; and
one or more organo-alkoxysiloxane monomers having the general formula Si(OR)₄₋ₓR′ₓ;
wherein:
   n is in the range of 0 to 100;
   x is in the range of 1 to 3;
   each of the R groups is selected from the group consisting of methyl, ethyl, propyl, isopropyl, and butyl alkyl; and
   each R′ is selected from the group consisting of methyl, ethyl, propyl, isopropyl, butyl, octyl, hexadecyl, octadecyl, phenyl, trimethylaminopropyl, aminopropyl, aminobutyl, methylphosphonyl, hydroxymethal, acetoxymethyl, acetoxypropyl, acetoxypolyethyleneoxypropyl, benzyl, carboxymethoxyethyl, expoxyhexyl, glycidoxypropyl, isocyanatopropyl, and phenethyl; and
wherein the one or more organo-alkoxysiloxane monomers are at an amount that is greater than 0 mol % and no more than about 30 mol % of the siloxane monomer component on a silicon mol basis.

In yet another embodiment, the present invention is directed to a method of hydrolyzing one or more acyl-homoserine lactones produced by one or more organisms of a gut microbiome within a gut of an individual. The method comprising administering an effective amount of the above-described stabilized enzyme product to the patient such that the stabilized enzyme product reaches the gut of the patient, and, while in the gut, the enzyme component remains active within the nanoporous organopolysiloxane matrix and hydrolyzes the one or more acyl-homoserine lactones produced by one or more organisms of a gut microbiome within the gut of the patient.

### BRIEF DESCRIPTION OF THE DRAWINGS

FIG. 1 is composite bar chart of the phylum level shallow metagenomics analysis of the microbiome from the mouse study using a DSS Crohns and Colitis model to introduce gut tissue damage.

### DETAILED DESCRIPTION OF INVENTION

The gut is home to a complex community of microbes that play important roles in the health of the host, from nutrition to disease prevention. The gut microbiome is so important that it is becoming recognized as a symbiont organ that is pivotal to life. The gut community represents a diverse microbiome that maintains a balance of organisms working together in their environment however within the community potential pathogens are present. Ordinarily, pathogenic members of the microbiome exist in low densities and are thought to be managed by a combination of host immune system and the microbiome community. Under conditions in which the gut becomes compromised either by traumatic injury or disease, the host immune system is weakened and microbiome responds to the changing conditions within the gut. As the gut enters a non-equilibrium state called dysbiosis, the relative populations within the microbiome may shift and overgrowth of potentially pathogenic microbes may become dominant. It is believed that alterations within the microbiome that favor potential pathogens complicate diseases associated with the gut leading to symptoms including fever, bloating, diarrhea, and abdominal pain. Chronic abdominal diseases such as irritable bowel syndrome, inflammatory bowel disease and Celiac's represent ongoing and progressive ailments that disrupt quality of life for the persons afflicted. Research focused on understanding and treating gut related diseases has been an ongoing and concerted effort which continues to accelerate in light of continued growth in the number of cases worldwide. More troubling is the broadening of patient categories including younger individuals and those from geographic regions with previously unreported incidents. What is known is that at least four factors contribute to disease development: diet, genetics, the immune system, and the gut microbiota. The gut microbiome is arguably the most complex and poorly understood factor associated with abdominal ailments and thus represents a largely untapped opportunity to create treatments for abdominal disease.

The present disclosure provides compositions and methods for delivering bioactive enzymes to at least a portion of the lower gut. The term “lower gut” may be used interchangeably with “lower gastrointestinal tract.” The lower gut is the segment of the gastrointestinal tract extending from the pyloric sphincter of the stomach of a human patient to the anus of the human patient. Typically, the lower gut consists of two segments, the small intestine and the large intestine. However, some human patients may have at least a portion of their lower gut surgically removed as part of a medical treatment.

The present invention is generally directed to enzyme-based products that are believed to interrupt chemical signals produced by bacteria responsible for inducing responses in other bacteria commonly known as quorum signaling. There are a number of microbial processes that are regulated by quorum signals, including chemo-attraction (swarming), virulence factor production, organization and formation of biofilms, induction of secondary metabolite production, and other metabolic functions. One of the more interesting aspects of quorum signaling-related processes is the manner in which chemical communication impacts complex microbial communities. Evidence to day suggests that population density and community diversity may be impacted by signaling between individuals within the microbiome, which are “informed” by the conditions within the local ecosystem.

More specifically, the present invention is believed to regulate quorum signaling through the use of homoserine lactonase enzymes, which reduce the intraluminal concentration of acylhomoserine lactone molecules thereby limiting quorum-based responses within the host gut microbiome. Results to do indicate that reducing the concentration of quorum signal molecules tends to reduce disease-related dysbiosis present in the gut, which includes limiting the overgrowth of certain phyla of bacteria. Within these phyla are species that have been correlated to a number of diseases that express specific symptoms. Managing microbiome dysbiosis via quorum signal concentration management can be used to reduce symptoms associated with abdominal distress thereby leading to improved quality of life for the host organism.

This disclosure provides compositions and methods which overcome at least some of the limitations associated with conventional synthesis methods for entrapping bioactive enzymes in an organosilicate gel matrix. In the present methods, a pre-polymer siloxane is prepared which is water-soluble. An aqueous enzyme solution is then added directly to the pre-polymer siloxane during gelation of the pre-polymer siloxane. In the present methods, the buffer contained within the enzyme solution is used as a catalyst of organosilicate gelation. Accordingly, catalysts and solvents which are typically used for siloxane polymerization in conventional methods are avoided in the presently described methods.

Advantageously, the resulting immobilized enzyme molecules are protected by the matrix from local environmental conditions including pH, and denaturants such as organic solvents and detergents. Further, the pore size of the matrix tends to be small enough such that protease enzymes cannot readily access and degrade the enzymes entrapped in the matrix. At the same time, the entrapped enzymes remain bioactive. Yet another technical advantage is that the entrapped enzymes tend to be too large to cross gut tissues barriers and thus do not absorb into the patient's blood and remain located in the lower gut until evacuated by bowel movement.

### Method of Producing a Stabilized Enzyme

The present method for stabilizing an enzyme include the following:
(a) hydrolyzing a siloxane monomer component of a hydrolysis reaction mixture, which also comprises an acid component and a solvent component, so that the siloxane monomer(s) of the siloxane monomer component form siloxane oligomer(s);
(b) removing any non-aqueous constituents from the solvent component from the product of the hydrolysis reaction (e.g., via evaporation);
(c) gelling the siloxane oligomer(s) with one or more enzymes to form a gel comprising sol-enzyme composites or particles, which comprise enzymes entrapped in nanoporous organopolysiloxane particles;
(d) curing the sol-enzyme composites to densify the matrix and stabilize the overall polysilicate structure;
(e) optionally removing or reducing the water content within the sol-enzyme composite via lyophilization to yield a low-density, friable powder that comprises particles of organopolysiloxane or silicate matrix with entrapped enzyme, wherein the silicate matrix has a BET surface area in a range of about 400 m²/g to about 600 m²/g;
(f) optionally grinding the sol-enzyme powder and/or classifying (e.g., via sieve(s)) the sol-enzyme powder to adjust the sizes of the powder particles to an average size of less than 50 micrometers;
(g) optionally removing unincorporated or exposed enzyme from the powder (e.g., by dispersing the powder in a solvent such as water and filtering the dispersed particles from the solvent so that the dissolved unincorporated or exposed enzyme is separated from the particles); and
(h) optionally the composite includes and acrylate polymer that imparts acid resistance (an enteric coating).

### I. Siloxane Monomer Component

As mentioned above, the siloxane monomer component is part of the hydration reaction mixture. A great variety of siloxane monomers or pre-polymers are suitable for inclusion as or part of the siloxane monomer component in the present enzyme entrapment method.

A. Tetraalkoxysiloxanes

The siloxane monomer component comprises one or more tetraalkoxysiloxane monomers having the general formula (I):

Si(OR)₄ (I);

wherein each of the R groups is selected from the group consisting of methyl, ethyl, propyl, isopropyl, and butyl alkyl. In one embodiment R is methyl. In another embodiment, R is ethyl. In yet another embodiment R is propyl.

B. Substituted Alkoxysiloxanes

It has been discovered that including alkoxysiloxanes in which one or more of the alkoxy groups is/are substitute with one or more functional groups may modify certain properties of the resulting oligomer that is to be mixed with enzymes to be gelled to form oganopolysiloxanes that have one or more certain desirable properties. In particular, tetraalkoxysilanes may be substituted with functional groups progressing from 4° for tetraalkoxysilanes to 3° for trialkoxysilanes, 2° for bialkoxysilanes, and 1° for monoalkoxysilanes.

1. Organo-alkoxysiloxanes

For example, the alkoxysiloxane monomer component may comprise one or more substituents selected from the group consisting of methyl, ethyl, propyl, isopropyl, butyl, octyl, hexadecyl, octadecyl, phenyl, trimethylaminopropyl, aminopropyl, aminobutyl, alkyl, mercaptopropyl, methylphosphonyl groups, hydroxymethyl, hydroxypropyl, methoxypropyl, benzyl, carboxymethoxyethyl, phenethyl, propylmethylcarbamate, and combinations thereof. These substituted alkoxysiloxanes may be referred to herein as “organo-alkoxysiloxanes” or “organo-siloxanes.” Experimental results to date suggest that including such organo-alkoxysiloxanes in the siloxane monomer component tends to enhance the stability of the enzyme(s) entrapped in the resulting silicate matrixes when exposed to the environment of the stomach. Also, the organic functional group(s) seem to stabilize the enzyme against denaturation during the gelation step described herein.

In one embodiment, the siloxane monomer component comprises one or more organo-alkoxysiloxanes having the general formula (II):

Si(OR)₄₋ₓR′ₓ (II);

wherein:
x is in the range of 1 to 3;
each R group is independently selected from the group consisting of methyl, ethyl, propyl, isopropyl, and butyl alkyl; and
each R′ is selected from the group consisting of methyl, ethyl, propyl, isopropyl, butyl, octyl, hexadecyl, octadecyl, phenyl, trimethylaminopropyl, aminopropyl, aminobutyl, methylphosphonyl, hydroxymethyl, hydroxypropyl, methoxypropyl, methoxymethyl, acetoxymethyl, acetoxypropyl, acetoxypolyethyleneoxypropyl, benzyl, carboxymethoxyethyl, expoxyhexyl, glycidoxypropyl, isocyanatopropyl, and phenethyl.

In another embodiment, x is 3, R is methyl, and R′ is phenyl. It yet another embodiment, x is 2, R is methyl, and R′ is methyl. In a further embodiment, x is 1, R is methyl, and R′ is methyl.

The results to date do not indicate any particular preferences among the same base alkoxysiloxane with different orders of organo substitutions. In other words, they may have substitution orders selected from the group consisting of 3°, 2°, 1°, or combinations thereof.

Results to date suggest that including organo-substitutions tends to increase the hydrophobicity of the resulting enzyme-silicate composite. Additionally, it has been observed that including organo-alkoxysiloxanes such that the siloxane monomer component has more than about 30 mol % of hydrophobic organic functional groups in the siloxane monomer component on a silicon mol basis tends to limit miscibility of the resulting enzyme-silicate composite particles in aqueous environments. Additionally, if the amount of ogano-substitutions in the siloxane monomer component is too great the resulting siloxane oligomer when mixed with the basic enzyme-containing solution will result in a product having an oil-like consistency not a firm gel.

In one embodiment, the amount of organo-alkoxysiloxanes in the siloxane component is between 0 mol % and about 30 mol % on a silicon mol basis. In another embodiment, the amount of organo-alkoxysiloxanes in the siloxane component is between 5 mol % and about 20 mol % on a silicon mol basis. In another embodiment, the amount of organo-alkoxysiloxanes in the siloxane component is between 5 mol % and about 15 mol % on a silicon mol basis.

An exemplary siloxane monomer component composition comprises: about 70 mol % Si(OR)₄, wherein R is ethyl, methyl, or propyl; about 20 mol % Si(OR)₃R′ wherein R is methyl, ethyl, or propyl, and R′ is phenyl; and about 10 mol % Si(OR)₃R′ wherein R is methyl, ethyl, or propyl, and R′ is trimethylaminopropyl.

In one embodiment, the amount of anionic organo-alkoxysiloxanes in the siloxane component is between 0.1 mol % and about 10 mol % on a silicon mol basis.

2. Anionic organo-alkoxysiloxanes

The siloxane component may further comprise anionic organo-alkoxysiloxane monomers, which are substituted with functional groups that introduce a negative charge in the matrices, and tends to make them useful as a buffering agent, enzyme stabilizer, and/or a dispersing agent. Anionic organo-alkoxysilanes also tend to stabilize the enzyme. Examples of such functional groups include thiols, esters of carboxylic acids (also referred to as carboxylic acid esters), sulfonates, and phosphonates. Exemplary thiol functional groups include mercaptopropyl, mercaptomethyl, and mercaptobutyl. An exemplary carboxylic acid ester is propylmethylcarbamate. An exemplary sulfonate is propane sulfonate. An exemplary phosphonate is propylmethylphosphonate. These anionic organo-alkoxysiloxanes tend to be 3° substituted siloxanes but may be 2° or 1° substituted siloxanes.

In one embodiment, the siloxane monomer component comprises one or more anionic organo-alkoxysiloxanes having the general formula (III):

Si(OR)₄₋ₓR″ₓ (III);

wherein:
x is in the range of 1 to 3;
each R group is selected from the group consisting of methyl, ethyl, propyl, isopropyl, and butyl alkyl; and
each R″ is selected from the group consisting of thiol, carboxylic acid ester, sulfonate, and phosphonate.

In one embodiment including an anionic organo-alkoxysilane in the siloxane monomer component, the selected anionic organo-alkoxysilane is such that x is 3, R is methyl, ethyl, or propyl, and R″ is propylmethylphosphonate. In another embodiment, x is 3, R is methyl, ethyl, or propyl, and R″ is propylmethylcarbamate. In yet another embodiment, x is 3, R is methyl, ethyl, or propyl, and R″ is mercaptopropyl, mercaptomethyl, or mercaptobutyl.

In one embodiment, the amount of anionic organo-alkoxysiloxanes in the siloxane component is between 0.1 mol % and about 10 mol % on a silicon mol basis.

3. Cross-Linking Organo-Alkoxysiloxanes

The siloxane component may further comprise cross-linking organo-alkoxysiloxane monomers, which are substituted with one or more cross-linking functional groups that may used to crosslink agents such as dyes that are used to track the final product in vivo (e.g., dye agents such fluorescein or rhodamine derivatives). Examples of such functional groups include glycidylpropyl, isocyanatopropyl, and cyanopropyl.

In one embodiment, the siloxane monomer component comprises one or more alkoxysiloxanes substituted with one or more cross-linking functional groups having the general formula (IV):

Si(OR)₄₋ₓR′″ₓ (IV);

wherein:
x is in the range of 1 to 3;
each R group is selected from the group consisting of methyl, ethyl, propyl, isopropyl, and butyl alkyl; and
each R′″ is selected from the group consisting of glycidylpropyl, isocyanatopropyl, and cyanopropyl.

In one embodiment, the amount of alkoxysiloxanes substituted with one or more cross-linking functional groups in the siloxane component is between 0.01 mol % and about 10 mol % on a silicon mol basis. In another embodiment, the amount of alkoxysiloxanes substituted with one or more cross-linking functional groups in the siloxane component is between 0.01 mol % and about 3 mol % on a silicon mol basis. In another embodiment, the amount of organo-alkoxysilanes in the siloxane component is between 0.01 mol % and about 0.1 mol % on a silicon mol basis.

Further, embodiments of various network polymers suitable for the present invention include those disclosed in U.S. Pat. Nos. 9,725,571; 6,033,781; and 5,993,967, each of which is incorporated herein by reference.

### II. Hydrolysis of Siloxane Monomer Component to Form a Sol

A. Hydrolysis Reaction—Acid Component

The siloxane monomer component is hydrolyzed under the acidic conditions in the pH range from about 1 to about 6. As indicated above, the hydrolysis is carried out in the presence of an acid component. The acid component may comprise one or more of nearly any type of acid, organic or inorganic. Exemplary acids include acetic, formic, sulfuric, hydrochloric, and combinations thereof.

In an embodiment, the acid(s) have an acid dissociation constant, pKa, below 4.

In one embodiment, the acid component consists of one or more organic acids selected from the group consisting of acetic acid, formic acid, and combinations thereof. In another embodiment, the acid component is acetic acid.

In another embodiment, the acid component consists of one or more inorganic acids selected form the group consisting hydrochloric acid, sulfuric acid, and combinations thereof. In yet another embodiment, the acid component is hydrochloric acid.

In one embodiment, the hydrolysis reaction mixture comprises an amount of the acid component that is in a range of about 1 mmolar about 100 mmolar.

B. Hydrolysis Reaction—Solvent Component

1. Water

The hydrolysis is accomplished with the siloxane monomer(s) dissolved or dispersed within a solvent component, which comprises water. In an embodiment, the water is at an amount a selected or regulated such that the resulting oligomers have a low-density (e.g., about 0.02 g/cm³ to about 0.5 g/cm³), a highly accessible surface area (e.g., about 250 m²/g to about 600 m²/g), fractal three-dimensional structure.

Without being bound to a particular theory, it is believed that the low-density, a highly accessible surface area, fractal three-dimensional structure results, at least in part, from the amount of water relative to amount of siloxane monomer(s). Preferably, the amount of siloxane monomers is quantified in terms the silicon content. In one embodiment, the mol ratio of water to silicon is in a range of about 0.2:1 to about 100:1. In another embodiment, the mol ratio of water to silicon is in a range of about 0.2:1 to about 20:1. In yet another embodiment, the mol ratio of water to silicon is about 0.2:1 to about 4:1.

2. Other Solvent(s)

While all hydration reactions are carried out in the presence of water and acid, some of the reaction solutions may also include an alcohol component, which comprises one or more suitable alcohols such as methanol or ethanol that act as a solvent for the siloxane monomer component or at least one of constituents thereof. Suitable alcohols include, but are not limited to, ethanol and methanol. Other polar protic solvents (e.g., propanol) may be used as well in addition or instead of ethanol and/or methanol.

If present, such non-aqueous solvent constituents are at an amount that is in a range of about 50% to about 98.8% of the solvent component.

C. Hydrolysis Reaction—Temperature

The hydration reaction may be carried at a temperature in the range from about room temperature (i.e., about 20° C.) to about 70° C. In an embodiment, the hydration reaction is carried out in the temperature range from about 40° C. to about 70° C. In another embodiment, the hydration reaction is carried out at 60° C.

D. Hydrolysis Reaction—Time

The time of the hydration reaction may selected or adjusted as needed in order to control, at least, the sizes of the aforementioned oligomers having a low-density, fractal three-dimensional structure. In an embodiment, the hydration reaction time is in a range of about 10 minutes to about 300 minutes. In another embodiment the hydration reaction time is in a range of about 30 minutes to about 120 minutes. In yet another embodiment, the hydration reaction time is in a range of about 60 minutes to about 90 minutes.

E. Control of Sol Polymeric Structure

The preparation of the matrix sol influences the overall performance of the composite in application. The control of the sol polymeric structure may be regulated by the initial hydrolysis conditions, including, for example, pH, water: silicon ratio, temperature and reaction time, which can lead to a myriad of structures from small linear oligomers to fractal three dimensional networks to aggregated nanoparticals. The preferred structure for the sol is a three dimensional fractal network, which provides rapid gelling and uniform entrapment of enzyme that, once gelled and cured, leads to a regular nano- and mesoporous structure in the final composite. The regular structure provides nano- and meso-channeling for ready access of fluid exchange necessary to access the entrapped enzyme.

### III. Solvent Exchange

The resulting oligomers are water miscible, which allows for solvent exchange (typically via evaporation) to eliminate any non-aqueous constituents of the solvent component or that were formed as side products such as alcohols. If desired, water may be added to the oligo-siloxane sol, for example, at an amount equal to the amount of alcohol removed. The resulting aqueous oligo-siloxane sol is ready for a base-catalyzed gelation as described in more detail below.

IV. Gelation of the Oligo-Siloxane Sol with One or More Enzymes

A gelation reaction mixture, which comprises the aqueous oligo-siloxane sol and an enzyme solution, is formed. The enzyme solution comprises an enzyme component. The gelation reaction mixture further comprises a base component and a buffer component, which may be added to the mixture individually, together, or as part of the enzyme solution. As such, the solution comprising the enzyme, base, and buffer components may referred to as a buffered basic enzyme solution. The base and buffer components catalyze the gelation of the oligo-siloxane sol thereby producing a gel that comprises a solid phase and a liquid phase, wherein the solid phase comprises enzymes entrapped in nanoporous organopolysiloxane.

Without being limiting, the following description is primarily directed to embodiments in which the enzyme solution comprises the base component and the buffer component, in addition to the enzyme component. That said, unless being expressly or impliedly limited to such multi-constituent enzyme solution embodiments, the follow description applies to embodiments in which the enzyme, base, and buffer components are individually or in various combinations mixed with the sol to form the gelation reaction mixture.

A. Enzyme Solution

1. Base Component

The enzyme solution has a basic pH due to the presence of a base component and/or a buffer component (below). It is believed that, if present, the base component affords a base catalyzed hydrolysis reaction that accelerates the siloxane gelation.

The pH of the gelation reaction mixture (in addition to amount of buffer component relative to the amount of siloxane oligomer) affects the gelation time. In one embodiment, both are controlled so that gelation occurs within five (5) minutes at about room temperature (20° C.).

In one embodiment the base component and its amount is selected such that pH of the gelation reaction mixture is in the range of about 7 to about 10. In another embodiment, the pH is in the range of about 7 to about 9. In yet another embodiment, the pH is about 8.

The base component may comprise one or more of nearly any type of base, organic or inorganic. That said, it is believed to desirable to not include inorganic mineral bases such as sodium hydroxide.

In an embodiment, the base(s) have an base dissociation constant, pKa, above about 8.0.

In one embodiment, the base component consists of one or more organic bases selected from the group consisting of alkyl substituted amines. In another embodiment, the base component is trimethyl amine.

In one embodiment, the base component consists of one or more organic bases selected from the group consisting of amine hydroxides. In another embodiment, the base component is ammonium hydroxide.

In another embodiment, the base component consists of one or more inorganic bases selected form the group consisting of alkali metal hydroxides. In yet another embodiment, the base component is sodium hydroxide.

The relative amounts of the base component and of the aqueous oligo-siloxane sol may be varied depending upon an application, the particular siloxane sol, and the particular enzymes. For convenience, the amount of oligo-siloxane is quantified in terms of the amount of siloxane monomer that was hydrolyzed to form the siloxane oligomer as described above. As such, in one embodiment, the molar ratio of siloxane monomer component to base component is in the range of about 1200:1 to about 50:1. In another embodiment, molar ratio of base component to siloxane monomer component is in the range of about 1000:1 to about 100:1. In yet another embodiment, molar ratio of base component to siloxane monomer component is in the range of about 800:1 to about 500:1.

2. Buffer Component

The enzyme solution may comprise a buffer component. In addition to buffering the pH of the enzyme solution, the buffer component may also serve as a catalyst for gelation. It is believed that, if present, the buffer component affords a base catalyzed hydrolysis reaction resulting in siloxane gelation. In one one embodiment, the buffer component is at a low concentration insufficient to increase the pH sufficiently to catalyze the gelation reaction and the enzyme solution also comprises the above-described base component. In another embodiment, the buffer component is at a concentration sufficient to increase the pH sufficiently to catalyze the gelation reaction.

Various buffers are suitable, including Tris buffers and/or Tris-phosphate buffers. Other exemplary buffers include phosphate and carbonates (bis, tris, and mes).

In addition to the pH, as mentioned above, the amount of buffer component relative to the siloxane oligomer(s) affects the gelation time. In one embodiment, both the base component and buffer component, if present, are selected such that that gelation occurs within five (5) minutes at about room temperature (20° C.).

The relative amounts of the buffer component and of the aqueous oligo-siloxane sol may be varied depending upon an application, the particular siloxane sol, and the particular enzymes. For convenience, the amount of oligo-siloxane is quantified in terms of the amount of siloxane monomer that was hydrolyzed to form the siloxane oligomer as described above. As such, in one embodiment, the molar ratio of siloxane monomer component to buffer component is in the range of about 100:1 to about 1:1. In another embodiment, molar ratio of buffer component to siloxane monomer component is in the range of about 80:1 to about 10:1. In yet another embodiment, molar ratio of buffer component to siloxane monomer component is in the range of about 50:1 to about 25:1.

3. Enzyme Component

Various enzymes may be entrapped accordingly to the present methods, including lactonases, organophosphate hydrolases, acylases and trypsin proteases. Other Gram positive and/or Gram negative quorum silencing enzymes and/or their combinations may be included as well. Quorum silencing enzymes in this disclosure include enzymes, catalytic domains of enzymes and enzyme derivatives which have been modified by recombinant technologies to, for example, improve stability, resistance to degradation, improved catalytic activity. Any enzyme that can be used to disrupt, at least partially, a signaling process in a bacterial population is referred to in this disclosure as a quorum silencing enzyme. More detailed information regarding specific enzymes for inclusion as the enzyme component or a portion thereof is set forth below.

In one embodiment, the enzyme solution comprises about 0.1% to about 50% of the enzyme component. In another embodiment, the enzyme solution comprises about 10% to about 40% of the enzyme component. In yet another embodiment, the enzyme solution comprises about 30% to about 40% of the enzyme component.

The relative amounts of the enzyme(s) and of the aqueous oligo-siloxane sol may be varied depending upon an application, the particular siloxane sol, and the particular enzymes. Typically, the amount of enzyme component relative to oligo-siloxane sol is selected to maximize its concentration such that a resulting powder or composition comprising said powder provides a reasonable potency of enzyme without destabilizing the integrity of the gel or matrix. In one embodiment, the amount of enzyme(s) is selected such that the enzyme component is at an mount that is in a range of about 0.01 wt % to about 40 wt % of the composite, on a dry weight basis.

For convenience, the amount of oligo-siloxane is quantified in terms of the amount of siloxane monomer that was hydrolyzed to form the siloxane oligomers as described above. As such, in one embodiment, the weight ratio of siloxane monomer component to enzyme component is in the range of about 1000:1 to about 1:1. In another embodiment, weight ratio of siloxane monomer component to enzyme component is in the range of about 500:1 to about 5:1. In yet another embodiment, weight ratio siloxane monomer component to enzyme component is in the range of about 100:1 to about 5:1.

B. Gelation Reaction—Temperature

The gelation reaction may be carried at a temperature in the range from about room temperature (i.e., about 20° C.) to about 60° C. In an embodiment, the gelation reaction is carried out in the temperature range from about 30° C. to about 50° C. In another embodiment, the gelation reaction is carried out in the temperature range from about 30° C. to about 40° C.

C. Gelation Reaction—Time

The gelation reaction is carried on for a period necessary for gelation to occur. The necessary time may depend upon a variety of parameters, including the temperature at which the gelation reaction is carried out and the composition of the oligo-siloxane sol.

In an embodiment, the gelation reaction time is in a range of about 5 minutes to about 120 minutes. In another embodiment the gelation reaction time is in a range of about 5 minutes to about 60 minutes. In yet another embodiment, the gelation reaction time is in a range of about 5 minutes to about 30 minutes.

### V. Curing the Gel

After the gelation is complete, the gel is cured to stabilize the gel which is demonstrated by condensation based syneresis of the solid gel form. Typical volumes for cured gels are 25% to 50% of the initial gel. The curing is conducted at appropriate temperature(s) for an appropriate duration. For example, gels comprising thermophilic enzymes may be cured at about room temperature or above whereas gels comprising mesophilic enzymes may be cured at temperatures less than room temperature but greater than 0° C. (e.g., 4° C.).

### VI. Drying the Sol-Enzyme Composite

Optionally, the cured gel is dried to remove or reduce the water content (e.g., via lyophilization) to yield a low-density, friable structure(s) that comprises and/or is readily reduced to powder-like particles (e.g., between about 1 micron and about 100 microns) that comprise enzyme component within a nanoporous oganopolysiloxane (or silicate) matrix.

### VII. Grinding the Dried Sol-Enzyme Composite

Optionally, the sol-enzyme powder may be subjected to grinding and/or classifying via any appropriate method/equipment (e.g., via ball mills, media mills, sieve(s), filters, classifiers, etc.) to adjust the size(s), average size, size range, etc. of the powder particles.

In order to maximize enzyme performance throughout the matrix, it is presently believed that the composite should be less than 100 micrometers in size. To provide low dust and formulation performance, the particles should preferably be greater than 1 micrometer in size. In one embodiment, the average particle size is about 10 micrometers.

### VIII. Removing Unincorporated Enzyme

Optionally, the sol-enzyme powder may be washed (pre-, post-, and/or with grinding) to remove or reduce the amount of unincorporated or exposed enzyme component from the powder. For example, this may be accomplished by dispersing the powder in a solvent such as water and filtering the dispersed particles from the solvent so that the dissolved unincorporated or exposed enzyme component is separated from the particles containing entrapped enzyme component.

Advantageously, the sol-enzyme powder may be dispersed in and separated from solvent such as water one or more time (i.e., “dehydrated” and “rehydrated”) without negatively impacting its performance. In contrast, previously known methods or forms for stabilizing such enzymes do not allow for rehydration.

The powder may be pressed into tablets and coated. Other oral formulations may be prepared as well, such as for example, a slurry and/or suspension, gel capsules, powder, pills, chewables or syrup.

### IX. Enteric Coating

Optionally, the particles of the sol-enzyme powder may further comprise an enteric coating applied by any appropriate method. The enteric coating may be an acrylate polymer such as Evonik's EUDROGIT. The coating is typically applied as a final step after any optional drying, grinding, sizing, and/or washing.

### X. Sol-Enzyme Powder Compositions

The sol-enzyme powder may be included in a variety compositions or formulations for suitable for delivery to a patient. For example, the sol-enzyme powder may be combined with an excipient, dispersant, flavoring agent, pH stabilizer, and/or a bulk filler. Pressed tablets (with or without coatings) and/or coated gel capsules may be prepared, according to any of the procedures typically used by a person of skill. Other compositions or formulations include slurries, suspensions, gel capsules, powder, chewables, syrups, etc. Other administration routes may include injections and direct delivery of a formulation to the lower gut, wherein the formulation is loaded on at least one medical device inserted into a patient. Accordingly, a formulation may be an injectable liquid formulation or powder formulation combined with a liquid excipient as needed prior to administration.

### XI. Enzymes

The action of enzymes to degrade quorum signaling molecules known as quorum quenching, which is also referred to as quorum silencing, provides a selective means to reduce concentration of these molecules in vivo. Enzymes capable of hydrolyzing quorum signaling molecules need to be protected from harsh environments and proteolytic degradation that occurs in the upper gut prior to delivery to site of action in the lower intestines. The above-described engineered silicate matrices immobilize quorum quenching enzymes and provide a platform by which these enzymes can be delivered to the microbiome while remain bioactive. The matrix entrapped enzyme used as the quorum silencer is stabilized against inactivation by denaturation or proteolytic digestion through the physical barrier, buffering and pore size characteristics of the inert silica scaffold. This quorum signal silencing strategy offers the advantage of acting at site of need without the risk of migration to the rest of the body via adsorption which may be the case for small molecule quorum inhibitors. Furthermore, the silica matrix protects the entrapped enzymes from degradation by gastric fluids.

Suitable enzymes for incorporating with the sol to form the above-described sol-enzyme composite include organophosphorus hydrolases that also have activity hydrolyzing acyl-homoserine lactones, which are quorum signaling molecules. Of particular interest are enzymes isolated from the following thermophiles strains:
Geobacillus stearothermophilus 10 (Gsp10);
Geobacillus stearothermophilus F(GspF); and
Sulfolobus solfataricus (Ssol), which is extremely thermophilic.
   Amino acid (protein) and the relevant DNA gene encoding sequence data for each enzyme is included below.

It is known that a conserved tryptophan residue at position 263 regulates protein flexibility, and contributes to catalytic efficiency and resistance to denaturation. Further, extensive mutagenesis of the tryptophan 263 in SSpox demonstrated that substitution with hydrophobic residues (particularly isoleucine) provided enhanced catalytic performance (in excess of 50×). Additionally, the literature contains extensive mutation studies involving the tryptophan residue for Ssol (SsolPox), as well as other sites, and it is disclosed that improved activity, in terms of kinetics and substrate specificity, can be ascribed to changing the tryptophan 263 to a hydrophobic residue. Billot et al., Engineering acyl-homoserine lactone-interfering enzymes toward bacterial control, J Biol Chem. (2020), 295(37), pp. 12993-13007. That said, it remains to be seen if the equivalent residue in Gsp10 or GspF affords similar results.

For reference, the tryptophan in question is identified with bold and italics in the sequence set forth below. The residues identified with bold and underline are four residues upstream in the sequence for SsolPox. The differences in the leading sequence may be strain dependent and believed to be insignificant because the activities of all the cited enzymes are essentially equivalent. The discrepancy may be the result of cloning issues reported by the Elias group in which an N-terminal tag was inserted and then removed post translation. The notable mutations are those that affect activity and improve catalytic rate, particularly at lower temperatures. For processing purposes both the Gs10 and GsF enzymes have been engineered with C-terminal poly His tags which are not removed and have not appeared to measurably impact enzyme properties.

The amino acid (protein) sequence of the Gsp10 enzyme, which is identified herein as SEQ ID NO:1, is as follows:

MAKTVETVLGPVPVEQLGKTLIHEHELFGYPGFQGDVTRGTFREDEALR
VAVEAAEKMKRHGIQTVVDPTPNDCGRNPAFLRRVAEETGLNIICATGY
YYEGEGAPPYFQFRRLLGTAEDDIYDMEMAELTEGIADTGIKAGVIKLA
SSKGRITEYEKMEFRAAARAQKETGAVIITHTQEGTMGPEQAAYLLEHG
ADPKKIVIGHMCGNTDPDYHRKTLAYGVYIAFDREGIQGMVGAPTDEER
VRTLLALLRDGYEKQIMLSHDTVNVWLGRPFTLPEPFAEMMKNWHVEHL
EVNIIPALKNEGIRDEVLEQMFIGNPAALFSAHHHHHH

The relevant DNA gene coding sequence of the Gsp10, which is identified herein as SEQ ID NO:2, is as follows:

ATGGCTAAAACTGTCGAAACCGTCCTTGGCCCAGTGCCGGTGGAACAGC
TTGGCAAAACGCTCATCCACGAGCATTTCCTCTTCGGCTATCCAGGGTT
TCAAGGCGATGTGACGCGCGGCACGTTCCGCGAAGACGAGGCGCTTCGC
GTCGCAGTCGAGGCGGCGGAAAAGATGAAGCGGCACGGCATTCAAACGG
TTGTCGATCCCACGCCGAACGACTGCGGGCGCAACCCGGCATTTTTGCG
GCGCGTTGCTGAAGAGACGGGACTGAACATTATTTGCGCCACCGGCTAT
TATTATGAAGGGGAAGGGGCGCCGCCGTACTTCCAATTCCGCCGGCTTC
TCGGAACAGCGGAAGATGATATTTACGACATGTTTATGGCCGAGCTGAC
CGAGGGCATTGCCGATACCGGAATCAAGGCGGGTGTCATCAAGCTCGCC
TCGAGCAAAGGGCGCATCACCGAGTACGAAAAGATGTTCTTCCGCGCTG
CCGCCCGCGCGCAAAAAGAGACGGGTGCGGTCATCATCACCCATACGCA
AGAAGGAACGATGGGGCCGGAACAAGCCGCCTATTTGCTTGAGCACGGC
GCCGATCCGAAAAAAATTGTCATCGGCCATATGTGCGGCAACACGGACC
CGGACTATCATCGAAAGACGCTTGCTTACGGCGTTTACATTGCGTTTGA
CCGCTTCGGCATCCAAGGGATGGTCGGCGCGCCGACTGATGAGGAGCGG
GTGCGGACGCTCCTTGCTCTGCTCCGCGATGGGTACGAGAAACAAATTA
TGCTGTCGCATGACACTGTCAACGTTTGGCTCGGTCGTCCGTTTACGCT
GCCGGAACCGTTTGCGGAAATGATGAAAAATTGGCATGTCGAGCATTTG
TTTGTGAACATCATCCCCGCGCTGAAAAATGAAGGAATCCGCGACGAAG
TGCTTGAGCAAATGTTCATCGGCAATCCGGCGGCGCTGTTCTCGGCTTG
A

The amino acid (protein) sequence of the GspF enzyme, which is identified herein as SEQ ID NO:3, is as follows:

MKKGMVETVCGPVPASELGKTLIHEHFVEGYPGFQGDVTLGPLRFEEAL
EAGIAVAQKMMAHGIQTVVDPTPNDCGRNPELLRRISEATGLNIICATG
YYYEGEGATPYFKFRRLLGTAEEEIYDMEMAEITNGIAGTGIKPGVIKL
ASSKNRITEYEKMEFRAAARVQKETGIVIITHTQEGTMGPEQAAYLLEH
GAVPQKIVIGHMCGNTDPEYHKKTLQYGVYIAFDREGLQGMVGAPTDEE
RIRTLLVLLREGFADKIMLAHDTVNIWLGRPLQLPEPFATMTKNWHVEH
VFEHIIPILTKEGVTEEQLEQMELKNPAALFASGHHHHHH

The relevant DNA gene coding sequence of the GsgF, which is identified herein as SEQ ID NO:4, is as follows:

ATGAAAAAAGGAATGGTGGAGACTGTCTGCGGCCCTGTGCCGGCGAGCG
AGCTTGGCAAAACGCTGATCCATGAGCATTTTGTCTTTGGGTATCCCGG
GTTTCAAGGCGATGTGACGCTCGGACCGCTTCGCTTTGAGGAAGCGCTC
GAGGCGGGAATTGCCGTCGCGCAAAAAATGATGGCCCATGGCATCCAAA
CCGTTGTCGATCCGACGCCGAATGACTGCGGCCGCAATCCGGAGCTGCT
CCGCCGCATTTCCGAAGCGACCGGGCTGAACATTATCTGTGCAACTGGA
TATTATTACGAAGGCGAAGGGGCAACGCCGTATTTTAAGTTTCGCCGGC
TGCTCGGTACGGCGGAAGAAGAAATATATGACATGTTTATGGCGGAAAT
TACAAACGGCATTGCCGGCACCGGCATTAAACCAGGCGTCATCAAACTG
GCGTCAAGCAAAAACCGGATCACCGAATATGAAAAAATGTTTTTCCGCG
CCGCGGCCCGCGTGCAAAAAGAAACAGGAATCGTGATTATCACCCATAC
GCAGGAAGGAACGATGGGGCCGGAACAGGCGGCATACTTGCTCGAGCAC
GGTGCCGTTCCGCAAAAGATTGTCATCGGCCATATGTGCGGAAACACCG
ATCCGGAATACCATAAAAAGACGCTGCAATACGGAGTGTACATCGCTTT
TGACCGCTTTGGCCTTCAAGGTATGGTCGGTGCCCCGACGGATGAAGAA
CGGATCCGCACGTTGCTCGTGCTGCTTCGCGAAGGCTTTGCTGATAAGA
TTATGCTCGCTCATGATACCGTCAATATTTGGCTCGGCCGTCCGCTGCA
GCTGCCAGAGCCATTTGCGACGATGACGAAAAACTGGCATGTCGAACAT
GTATTTGAACATATTATTCCAATTTTAACGAAAGAAGGAGTGACAGAGG
AACAGCTAGAGCAAATGTTTTTGAAAAATCCTGCCGCTTTGTTTGCG

The amino acid (protein) sequence of the Ssol enzyme, which is identified herein as SEQ ID NO:5, is as follows:

MRIPLVGKDSIESKDIGFTLIHEHLRVFSEAVRQQWPHLYNEDEEFRNA
VNEVKRAMQFGVKTIVDPTVMGLGRDIREMEKVVKATGINLVAGTGIYI
YIDLPFYFLNRSIDEIADLFIHDIKEGIQGTLNKAGEVKIAADEPGITK
DVEKVIRAAAIANKETKVPIITHSNAHNNTGLEQQRILTEEGVDPGKIL
IGHLGDTDNIDYIKKIADKGSFIGLDRYGLDLELPVDKRNETTLRLIKD
GYSDKIMISHDYCCTIDWGTAKPEYKPKLAPRWSITLIFEDTIPFLKRN
GVNEEVIATIFKENPKKEFS

The relevant DNA gene coding sequence of the Ssol, which is identified herein as SEQ ID NO:6, is as follows:

xxxxxxxxxxxxatgagaataccattagttgggaaagattcaatagaat
ctaaggacataggatttacgctaattcatgaacatttaagagtttttag
cgaagcggtcagacaacaatggccccatctatataacgaagatgaggag
ttcagaaacgctgtaaatgaggttaaaagggcaatgcaatttggagtaa
agactatagtagatcccactgtaatgggattgggtagggacatcagatt
tatggaaaaagtggttaaggctaccgggataaatttagttgcggggacg
gggatttacatatatatcgacttacctttctatttcttaaataggtcaa
ttgatgagatagctgacttgtttattcatgatataaaagagggaataca
aggtactctcaataaagctggcttcgtaaagatagctgcagatgaacct
gggatcacaaaggatgtggagaaggtaataagggctgctgccatagcaa
acaaagagactaaagtaccaataattacccactctaacgctcacaataa
caccggattagaacagcaaagaatattgactgaagaaggtgttgatcca
gggaaaatattaataggtcatttaggtgatacagataatatagattaca
taaagaagatagcagataagggatcctttattggattagatagatatgg
tttagatttattcctacctgttgataagagaaatgaaacgaccttaaga
ctaatcaaagatggttattcagataagataatgatctctcacgattatt
gttgcacaatcgactggggaactgcaaaaccagaatataaacctaagct
tgctccaagatggagtataactctaatatttgaggatacgataccgttc
ttaaagagaaatggagtgaatgaagaggttatagctacaatatttaagg
aaaatccgaaaaagttcttcagctaa

The amino acid (protein) sequence of the SsolPox enzyme, which is identified herein as SEQ ID NO:7, is as follows:

MRIPLVGKDSIESKDIGETLIHEHLRVFSEAVRQQWPHLYNEDEEFRNA
VNEVKRAMQFGVKTIVDPTVMGLGRDIREMEKVVKATGINLVAGTGIYI
YIDLPFYFLNRSIDEIADLFIHDIKEGIQGTLNKAGEVKIAADEPGITK
DVEKVIRAAAIANKETKVPIITHSNAHNNTGLEQQRILTEEGVDPGKIL
IGHLGDTDNIDYIKKIADKGSFIGLDRYGLDLFLPVDKRNETTLRLIKD
GYSDKIMISHDYCCTIDIGTAKPEYKPKLAPRWSITLIFEDTIPELKRN
GVNEEVIATIFKENPKKEFS

The relevant DNA gene coding sequence of the SsolPox, which is identified herein as SEQ ID NO:8, is as follows:

atgagaatac cattagttgg gaaagattca atagaatcta
aggacatagg atttacgctaattcatgaac atttaagagt
ttttagcgaa gcggtcagac aacaatggcc
ccatctatataacgaagatg aggagttcag aaacgctgta
aatgaggtta aaagggcaat gcaatttggagtaaagacta
tagtagatcc cactgtaatg ggattgggta gggacatcag
atttatggaaaaagtggtta aggctaccgg gataaattta
gttgcgggga cggggattta catatatatcgacttacctt
tctatttctt aaataggtca attgatgaga tagctgactt
gtttattcatgatataaaag agggaataca aggtactctc
aataaagctg gcttcgtaaa gatagctgcagatgaacctg
ggatcacaaa ggatgtggag aaggtaataa gggctgctgc
catagcaaacaaagagacta aagtaccaat aattacccac
tctaacgctc acaataacac cggattagaacagcaaagaa
tattgactga agaaggtgtt gatccaggga aaatattaat
aggtcatttaggtgatacag ataatataga ttacataaag
aagatagcag ataagggatc ctttattggattagatagat
atggtttaga tttattccta cctgttgata agagaaatga
aacgaccttaagactaatca aagaatttta ttcagataag
ataatgatct ctcacgatta ttgttgcacaatcgactggg
gaactgcaaa accagaatat aaacctaagc ttgctccaag
atggagtataactctaatat ttgaggatac gataccgttc
ttaaagagaa atggagtgaa tgaagaggttatagctacaa
tatttaagga aaatccgaaa aagttcttca gctaa

The enzymes were cloned and overexpressed with codon optimization for E. coli along with a C-terminal 6×His tag as an aid for enzyme purification. Further, the Gsp10 enzyme was successfully expressed, purified characterized, and crystallized. The Gsp10 protein structure was solved to a 2 Å resolution. GspF was expressed, purified, and characterized but its structure was not determined. SsolPox was cloned, expressed, and partially characterized as well as preliminary crystallization performed, however, the enzyme was difficult to express with a reasonable yield and the kinetics were found to be highly temperature dependent with low activity near ambient conditions.

### XII Stabilized Enzyme Product

Conducting the above-described method results in a stabilized enzyme product comprising composite particles that comprise an enzyme component within a nanoporous organopolysiloxane matrix, wherein the enzyme component is capable of hydrolyzing one or more acyl-homoserine lactones. In other embodiments, the enzyme component comprises a sequence selected from the group consisting of SEQ ID NO: 1, SEQ ID NO: 3, SEQ ID NO: 5, and SEQ ID NO: 7, and wherein the nanoporous organopolysiloxane matrix has a density in a range of about 0.02 g/cm³ to about 0.5 g/cm³, an accessible surface area in a range of about 250 m²/g to about 600 m²/g, and fractal three-dimensional structures. As mentioned above, the nanoporous organopolysiloxane is derived from the above-described siloxane monomer component.

### XIII Diseases and Treatments

Various diseases and disorders, including general gut discomfort, may be treated with formulations comprising quorum silencing enzymes. The present formulations comprising quorum silencing enzymes entrapped in the organopolysiloxane matrix may be used to treat an individual or patient whose gut microbiome is abnormal. Such individuals or patients include those who are diagnosed with the gut microbiome dysbiosis. The term “gut microbiome dysbiosis” is used in this disclosure broadly and includes any condition under which there is an imbalance in the gut microflora. The gut microbiome dysbiosis may include an overgrowth of bacteria and/or yeast strains. Other causes of the imbalance in the gut microflora include an undergrowth of gut bacteria and/or yeast and/or a growth of bacteria and/or yeast in the gut area such as small intestine. A patient with the gut microbiome dysbiosis may be treated with a formulation comprising one or more quorum silencing enzymes entrapped in the organopolysiloxane matrix, as provided in this disclosure.

Diseases that can be treated with the formulations of the present disclosure include, but are not limited, to intestinal inflammatory diseases and/or any other diseases, symptoms of which may be ameliorated by improving the balance in the gut microbiome. Intestinal inflammatory diseases that can be treated with the formulations include inflammatory bowel disease, Celiac disease, gluten intolerance, ulcerative colitis and Crohn's disease. Other intestinal diseases include colon cancer and any other intestinal cancers. Stated another way, an abnormal gut microbiome may be treated. An “abnormal gut microbiome” is understood broadly and includes any imbalance in the gut microbiome, such as a change in microbial species and/or a change in a number (a decrease or increase) and/or a growth in the gut areas not commonly occupied by microflora. The abnormal gut microbiome may be tested via a number of tests commonly known to a medical practitioner, for example, a microflora from a stool sample may be cultivated and analyzed for microbial species and a number.

Methods of treatment include administering to a patient in need of treatment for abnormal gut microbiome, a formulation comprising one or more quorum silencing enzyme entrapped in the organopolysiloxane matrix, obtained as described in this disclosure.

The formulation may be administered as an oral formulation and/or as a rectal suppository, by enema, by direct injection or delivered directly to the lower gut with by a medical device. The one or more quorum silencing enzymes entrapped in the organopolysiloxane matrix are administered in an effective amount which is any amount sufficient to at least partially decrease inflammation in the lower gut and/or at least partially silence a signaling between bacteria at least in a portion of the patient's lower gut.

In some treatment methods, the effective amount of a bioactive enzyme may be any amount in the range from about 0.5 mg to 200 mg of the bioactive enzyme per one dosage if formulated as a gel capsule. Tablets may have effective bioactive enzyme dosages from 0.5 mg to 800 mg per dosage. Chewable formulations may have dosages from 0.5 mg to 200 mg per dosage. Liquid suspensions may have dosages from 0.5 mg per mL to 50 mg per mL per dosage. The total effective amount administered per day may vary, depending on various factors, including the patient's condition, weight and response to the treatment. A dosage may be increased or decreased, as needed.

Methods of treatment include administering to a patient in need of a treatment for an inflammatory intestinal disease, an oral formulation and/or a formulation administered as a rectal suppository or by enema, the formulation comprising one or more quorum silencing enzyme entrapped in the organopolysiloxane matrix, obtained as described in this disclosure.

### EXAMPLE

### Production of JLB-1061

Preparation of prototype OPH/polysiloxane composites. Prior methods of of preparing composites suggest the potential feasibility of silicate enzyme composites that display stability and retain catalytic activity. Ellerby et al., Encapsulation of proteins in transparent porous silicate glasses prepared by the sol-gel method, Zink JI Science, 1992, Feb. 28, 255(5048), pp. 1113-5; Massari et al., Dynamics of Proteins Encapsulated in Silica Sol-gel Glasses Studied with IR Vibrational Echo Spectroscopy; Journal of the American Chemical Society, 2006, 128(12), pp. 3990-3997; Calabretta et al., Silica as a Matrix for Encapsulating Proteins: Surface Effects on Protein Structure Assessed by Circular Dichroism Spectroscopy, Journal of Functional Biomaterials, 2012, 3(3), pp. 514-527. Past efforts, although demonstrative, suffered from key limitations that preclude consideration for broad applicability. Most notably, the present inventor(s) found that the process for immobilizing the enzyme is difficult to scale owing to the delicate balance of forming a stable silicate precursor emulsion in large volumes. Furthermore, if not treated with caution, prior examples of enzymes immobilized in silicate supports rapidly lost activity if the support was allowed to dry. Finally, as with many simple aerogels and xerogels, the structural stability of the silicate was sensitive to damage via hydraulic pressure shock as liquid gradients form across meso and micro channels in the material.

To address these issues, the present process involves preparing a pre-polymer siloxane and uses an in situ solvent exchange process to enable soluble single phase siloxane/enzyme solutions to be mixed prior to cure. The siloxane polymer was designed and engineered to match effective surface dielectric surfaces on the enzyme to help stabilize charged and nonpolar environments needed for consequent catalytic activity. As such, the enzyme silicate composite may be dried by lyophilization to form a stable powder that may be rehydrated while maintaining enzyme stability and catalytic activity within about 80% of the starting enzyme stock solution.

A laboratory experiment was performed by adding 6.8 grams of tetraethoxysilane (0.033 mol), 1.5 grams of methyltrimethoxy silane (0.005 mol), and 0.9 grams of trimethylammoniumtrimethoxypropylsilane (0.005 mol) to stirred flask containing 20 ml of ethanol. The reaction was started by addition of 200 microliters deionized water and 200 microliters 0.1M hydrochloric acid followed by raising the temperature to 60° C. The reaction proceeded for 1 hour following which 25 ml deionized water was added and vacuum applied to remove the alcohol solvent. The temperature was reduced to ambient and 5 ml of a 50 mg/ml stock solution of Geobacillus OPH in 01.M pH 8 Tris buffer was added. The reaction was allowed to stand at room temperature until gelation occurred. The sample was allowed to cure overnight at room temperature and then frozen at −80° C. The frozen sample was lyophilized affording a tan powder. Enzyme assays described above substituting the powder demonstrated the material maintained catalytic activity via TLC analysis of 3-oxo-C(12)-HSL.

### In Vitro Testing of JLB-1061

OPH Catalyzed Degradation of 3-oxo-C(12)-HSL. AHLs are not widely available commercially and, as such, C(4)-HSL and 3-oxo-C(12)-HSL were synthesized according to Chhabra et al., J Med Chem, 2003, 46, pp. 97-104 with modifications. The purified substrate was dissolved in a stock solution of acetonitrile and diluted to final concentration of 10 mM in 50 mM Tris buffer pH 7.4. The reactions were initiated by adding purified OPH prepared in a stock solution of 50 mM tris buffer pH 7.4 to a final concentration of 10 micrograms per milliliter. The reactions were run in a shaking incubator at 30° C. for 10 hours. Progress of the reaction was monitored by thin layer chromatography. Water from the reactions was removed in vacuo to afford a residue, which was dissolved in acetonitrile. The precipitated protein and buffer salts were removed by filtration. Identification of reaction products was accomplished by LC-MS using a C18 reverse phase column. A single product was recovered from the reaction mixtures corresponding to 3-oxo-N-octadecanoylhomoserine, corresponding to the presence of lactonase enzyme activity, which was not formed in the absence of the OPH.

Inhibition of QS-dependent Pigment Formation of Chromobacterium violaceum. A bioassay was designed based on the known QS process for the formation of the C. violaceum purple pigment violacein. Lichstein et al., Violacein, an antibiotic pigment produced by Chromobacterium violaceum, J. Infect. Dis., 1945, 76, pp. 47-51. To this end, cultures of C. violaceum were grown in LB medium at 37° C. in a 24 well microtiter plate overnight. Sterile filtered purified GspF OPH(His₆) was added exogenously to growing cultures at concentrations ranging from 1 μg/ml to 1 mg/ml. Control cultures in which the GspF OPH(His₆) was replaced with BSA, or lacking any additional protein were also conducted. The 0.1 mg/ml GspF OPH(His₆) was able to inhibit C. violaceum purple pigment formation, suggesting that it can degrade N-hexanoyl-L-homoserine lactone. Inhibition of pigment formation by GspF OPH(His₆) was concentration-dependent. After completion of the assays, enzyme activities in the cell free spent culture broths were tested using methyl paroxon and coumaphos as substrates. OPH enzyme catalytic activity remained in cell free culture broths, demonstrating the durability of this enzyme. No hydrolytic activity was observed for cell-free culture broths without added OPH enzymesGspF OPH(His₆) stock enzyme.

AHL-ase mediated Inhibition of QS-dependent Biofilm Formation. P. aeruginosa PA01 was used to assay for the effect of exogenously added AHL-ase enzymes on biofilm formation in liquid culture. Biofilm formation of PA strain PA01 has been well characterized. Saiman et al., Comparison of adherence of Pseudomonas aeruginosa to respiratory epithelial cells from cystic fibrosis patients and healthy subjects, Infect Immun (1992), 60(7), pp. 2808-14; Mathee et al., Mucoid conversion of Pseudomonas aeruginosa by hydrogen peroxide: a mechanism for virulence activation in the cystic fibrosis lung, Microbiology (1999), 145(Pt 6), pp. 1349-57; Rodgers et al., In vitro susceptibility testing of topical antimicrobial agents used in pediatric burn patients: comparison of two methods, J Burn Care Rehabil (1997), 18, pp. 406-410. Biofilm assays were performed with minor modifications according to literature precedent. Pratt et al., Mol Microbiol (1998), 30, pp. 285-293. A 3-hour pre-culture of P. aeruginosa was followed by addition of filter sterilized AHL-ase stock enzyme. Wells were washed with PBS buffer and biofilms were stained with crystal violet. The presence of biofilms was determined by the absorbance of remaining crystal violet at 540 nm and the data recorded as a percent of inhibition of biofilm as shown in Table 1 below. Hentzer et al., Microbiology (2002), 148, pp. 87-102. Substitution of BSA for ALH-ase in equivalent assays demonstrated no biofilm inhibition. The apparent saturation of biofilm inhibition is believed to result from cell adhesion during the early pre-culture phase of the experiment. Further biofilm formation studies are ongoing to address experimental design issues that lead to early cell adhesion which impacts AHL-ase assays.

TABLE 1

| Gs-FAHL-ASE(His6) | % Biofilm |
|---|---|
| Concentration (mg/mL) | Inhibition |
| 0 | 0 |
| 0.001 | 23 |
| 0.01 | 35 |
| 0.1 | 52 |
| 1 | 54 |

Table 1 contains values for percentage of remaining biofilm were determined from the absorbance difference between control and AHLase containing cultures. All experiments were run in triplicate and are corrected for absorbance due to nonspecific dye binding to the surface of the microtiter tray.

### In Vivo Testing of JLB-1061

A well-established in vivo mouse model was used to evaluate JLB-1061 to test how enzyme activity could impact animal health and the gut microbiome. In particular, the DSS Crohns and Colitis model was selected for testing not because of its relevance to Crohns and colitis but the model's reliable and understood mechanism for introducing gut tissue damage. The DSS protocol is a common model used for studying Crohns and colitis and is well understood at the histologic an immunologic level (see Wirtz et al., Nature Protocols, 2017, 12(7), p. 1295). Furthermore, an increasing number of peer reviewed papers use the DSS model to characterize changes at the microbiome level and provide correlations between observed dysbiosis and disease progression. The DSS model was adapted to introduce the solid JLB-1061 in slurry form at high doses to test for material toxicity and to gauge the effect on disease progression and microbiome population dynamics.

The testing followed the general 9 day protocol which introduced DSS reagent into the subject drinking water over five days. Controls included healthy mice, mice exposed to DSS alone, mice exposed to DSS and treated with JLB silicate without AHLase. A test population (10 mice each) was treated with JLB-1061 twice daily at Day 1 continuing through Day 9 and a separate population of subjects (10 mice) were treated with JLB-1061 twice daily beginning at Day 5 continuing through Day 9. Daily evaluation of animal health was determined by measuring weight, activity and stool quality. Surviving animals were sacrificed, final stool samples collected and colons resected and prepared for histologic examination. Stool samples at Day 3 and Day 9 were prepared for metagenomics analysis and forwarded to an independent laboratory for testing using an Illumine system and the data was analyzed using the One Codex database.

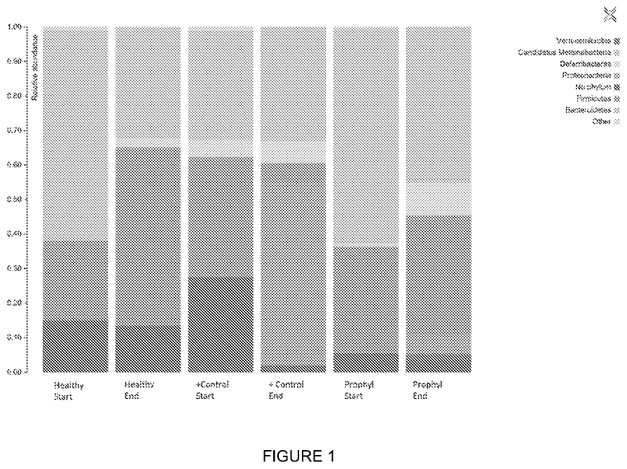

Referring to FIG. 1, the chart represents normalized data to quantify the relative populations of microbes found within the gut based on database identification using the One Codex platform. Positive control mice receiving DSS show a significant increase in Proteobacteria which is consistent with peer reviewed literature. Additionally, and also consistent with literature, tissue damage to the gut results in a dysbiotic microbiome that displays a reduced diversity at the microbial species level. Healthy mouse control subjects display aging related changes to the microbiome as evidenced by the proportional increase in Firmicutes. Test subjects receiving JLB-1061 prophylactically showed modest changes in microbiome composition at the phylum level compared to positive control subjects. JLB-1061 prophylactic treatment subjects showed increases in Deferribacteres, a modest emergence of Proteobacteria, and altered ratio of Bacteroidetes to Firmicutes. At the species level, there is some reduction in species diversity which appears to correlate with shifts in phylum level analysis. Importantly, gross evaluation of animal health in terms of behavior (i.e., grooming, activity, and appetite), weight, and stool quality all appeared to suggest subjects were generally healthier than DSS treated subjects. Interestingly, histologic analysis of colons suggested that significant tissue damage was present in all DSS treated subjects, including JLB-1061 treated subjects. Thus, the data suggests that although there was substantial colon tissue damage, the microbiota did not generally present substantial runaway dysbiosis as evidenced by a lack of Proteobacteria bloom.

While the One Codex platform is the most current and curated database for microbiome characterization, the sheer complexity of the gut microbiome is reflected in the significant number of metagenomics “reads” that fall into uncharacterized pool. Despite the limitations associated with the analysis, the metagenomics analytical tool provides the best overview of speciation within the gut microbiome. To this end, the peer reviewed literature and metagenomics analysis provide sufficient basis to enable testing of microbiome behavior in response to quorum signal concentration management.

The JLB-1061 composite drove AHLase activity impacting specific population dynamics within the gut microbiome preventing overgrowth/dysbiosis. As such, the JLB-1061 composite may be used to manage symptoms associated with abdominal distress. As such, it is believed that JLB-1061 may have applications to a number of disease states and other digestive disorders in which acute or chronic dysbiosis may lead to discomfort and opportunistic infection therefore leading to complications that may delay patient recovery, psychology and ultimately quality of life outcomes.

The observation that microbes in the Proteobacteria genus did not overgrow in treated disease subjects could be inferred based on above-described respiratory in vivo research. However, the lack of significant impact on other microbial genus populations is unexpected. Further, the observation of low disease indices particularly for prophylactically treated mice including relatively healthy behavior in spite of clear histological demonstration of tissue damage was unexpected. The in vivo data suggests that the JL-1061 had the following effects:
suppression of Proteobacteria overgrowth, which ordinarily results in a dysbiotic microbiome;
improved disease index, in spite of demonstrated tissue damage;
prevention of disease-related physical symptoms, as defined by healthy behavior in spite of demonstrated tissue damage; and
prophylactic treatment with JL-1061 before disease induction resulted in a significantly greater effect compared to treatment immediately after disease induction.

As a result, it is believed that the compositions of the present invention offer a different approach to gut intervention than typical treatments such as those targeting the microbiome (e.g., antimicrobials, fecal cell transplants) or host tissue damage (e.g., immune suppression, anti-inflammatories). As mentioned above, the present approach is directed AHLase dependent microbiome responses rather than antimicrobial and anti-inflammatory, and compositions of the present invention may find value not only as a stand-alone product but also in compliment to other treatments listed above as well as diet based therapies.

The metagenomics data set may be further articulated at the species level, which provides intriguing insights into the phenomena of eliminating acylhomoserine lactones from the gut lumen. For example, it was observed:
healthy subject controls displayed a shift in microbiome community to favor the genus Firmicutes over Bacteroides with no significant change in Proteobacteria, which is consistent with the literature;
there was an emergence of Porphyronnodaceae as well as Lachnospiraceae; and
losses in Bacteroides sps. and Parabacteroides are made up as the mice age.
   Also, in agreement with literature, the positive controls consistently displayed the following:

the Proteobacteria overgrew the microbiome, with E. coli, Shigella sp. and Clostridia sp. making up much of the genus;
there was a loss of microbiome diversity at the species level;
the changes in species variation did not appear consistent subject-to-subject, meaning that in some cases microbes in the Firmicutes genus were lost, while in other cases species within Bacteroides were compromised; and
in all positive controls, Mucispirillum schaedleci from the Deferribacteres genus remained consistently present.
   The subjects that received the JLB-1061 composite, whether treating prophylactically or post disease induced, the following was observed:
while present, E. coli and Clostridia sp did not overgrow and Clostridia sp. remained a small population;
in most subjects, the speciation diversity was reduced, however, not to the same degree as observed in the positive controls;
there was a shifting of species within Bacteroides and Firmicutes genus', however, the relative populations and specific species did not show consistent trends; and
Porhyomonadaceae generally became more prominent in prophylactically treated mice.

Evaluating and comparing the prophylactically-treated subjects to the post-disease induced subjects at the species level of metagenomics analysis did not clearly demonstrate the observed differences in disease index or animal behavior. The prophylactically treated had healthier behaviors and more favorable disease indices. It is consistent that microbiome diversity is more negatively impacted in post-disease induction treatments as well as potentially the presence of a higher percentage of Proteobacteria. The observations can be explained by the timing of dysbiosis onset in that Proteobacterial emergence occurs during disease induction. As such relative population shifts once induced will persist but can be limited in scale with treatment with JL-1061. Prophylactic treatment appears to prevent the dysbiosis emergence and controls Proteobacteria overgrowth throughout the test regimen. With regard to host subject tissue damage, the histology analysis does not significantly explain the differences between the two treatment groups; both sets of subjects have essentially equivalent histology results.

The term “about” is to be construed in accordance with the understanding of a person of ordinary skill in the art. That said, if there is doubt as to such a construction, the term “about” is to be construed, when used in relation to a number, to be minus a certain percentage of said number, plus a certain percentage of said number, or plus and minus a certain percentage of said number, depending upon the context. For example, if said number is the lower value of a range, one would understand that the “about” extends the range minus or below said number by a certain percentage. Conversely, if said number is the upper value of a range, one would understand that the “about” extends the range plus or higher than said number by a certain percentage. And if the number is not a lower or upper value of a range, one would understand that the “about” creates a range about said number with the lower value of the range corresponding to the certain percentage minus or below said number and the upper value of the range corresponding to the certain percentage plus or higher than said number. In reference to this disclosure, the “certain percentage” referred to herein is 20%, 15%, 10%, or 5%.

Having illustrated and described the principles of the present invention, it should be apparent to persons skilled in the art that the invention can be modified in arrangement and detail without departing from such principles.

Although the materials and methods of this invention have been described in terms of various embodiments and illustrative examples, it will be apparent to those of skill in the art that variations can be applied to the materials and methods described herein without departing from the concept, spirit and scope of the invention. All such similar substitutes and modifications apparent to those skilled in the art are deemed to be within the spirit, scope and concept of the invention as defined by the appended claims.

1. A method of stabilizing an enzyme, the method comprising:
forming a hydrolysis reaction mixture that comprises a siloxane monomer component, an acid component, and a solvent component, wherein:
the acid component is at an amount sufficient for the hydrolysis reaction mixture to have a pH in a range of about 1 to about 6 and to catalyze the hydrolysis of the siloxane component thereby producing an oligo-siloxane sol that comprises siloxane oligomers;
the solvent component comprises water at an amount such that a mol ratio of water to silicon of the siloxane monomer component is in a range of about 0.2:1 to about 100:1; and
the siloxane monomer component comprises:
one or more tetraalkoxysilane monomers having the general formula Si(OR)₄ or (OR)₃Si(OSi(OR)₂)ₙOSi(OR)₃; and
one or more organo-alkoxysiloxane monomers having the general formula Si(OR)₄₋ₓR′ₓ;
wherein:
n is in the range of 0-100;
x is in the range of 1 to 3;
each of the R groups is selected from the group consisting of methyl, ethyl, propyl, isopropyl, and butyl alkyl; and
each R′ is selected from the group consisting of methyl, ethyl, propyl, isopropyl, butyl, octyl, hexadecyl, octadecyl, phenyl, trimethylaminopropyl, aminopropyl, aminobutyl, methylphosphonyl, hydroxymethyl, hydroxypropyl, methoxypropyl, methoxymethyl, acetoxymethyl, acetoxypropyl, acetoxypolyethlyeneoxypropyl, benzyl, carboxymethoxyethyl, epoxyhexyl, glycidoxypropyl, isocyanopropyl, and phenethyl; and
wherein the one or more organo-alkoxysiloxane monomers are at an amount that is greater than 0 mol % and no more than about 30 mol % of the siloxane monomer component on a silicon mol basis;
removing any non-aqueous solvents from the oligo-siloxane sol to form an aqueous oligo-siloxane sol;
forming a gelation reaction mixture that comprises the aqueous oligo-siloxane sol, an enzyme component, and a base component and/or a buffer component, wherein the gelation reaction mixture has a pH in a range of about 7 to about 10, wherein the base component and/or the buffer component catalyze the gelation of the aqueous oligosiloxane sol thereby producing a gel that comprises a solid phase and liquid phase, wherein the enzyme component is capable of hydrolyzing one or more acyl-homoserine lactones, wherein the solid phase comprises the enzyme component entrapped in nanoporous organopolysiloxane, and wherein the entrapped enzyme component constitutes about 0.01 wt % to about 40 wt % of the solid phase on a dry weight basis; and
curing the gel to form a solid hydrogel;
thereby stabilizing the enzyme.

2. The method of claim 1, wherein the siloxane oligomers have three-dimensional fractal structures.

3. The method of claim 1, wherein:
for the one or more tetraalkoxysilane monomers, R is methyl, ethyl, or propyl; and
for the one or more organo-alkoxysiloxane monomers, x is 2 or 3, R is methyl or ethyl, and R′ is methyl or phenyl.

4. The method of claim 1, wherein the amount of the organo-alkoxysiloxanes in the siloxane monomer component is in a range of about 5 mol % to and about 20 mol % on a silicon mol basis.

5-16. (canceled)

17. The method of claim 1, wherein the acid component is selected from the group consisting of sulfuric acid, formic acid, acetic acid, and hydrochloric acid, and wherein the acid component is at an amount of about 1 mmolar to about 100 mmolar of the hydrolysis reaction mixture.

18. (canceled)

19. (canceled)

20. The method of claim 1, wherein the amount of water in the hydrolysis reaction mixture is such that the mol ratio of water to silicon of the siloxane monomer component is in a range of about 0.2:1 to about 4:1.

21. The method of claim 1, wherein the solvent component further comprises one or more non-aqueous constituents at amount that is in a range of about 50% to about 98.8% of the solvent component, and wherein the non-aqueous constituents of the solvent component are one or more alcohols.

22. (canceled)

23. The method of claim 1, wherein the enzyme component comprises SEQ ID NO: 1, SEQ ID NO: 3, SEQ ID NO: 5, or SEQ ID NO: 7.

24. (canceled)

25. (canceled)

26. (canceled)

27. The method of claim 1 further comprising:
reducing the water content of the cured gel to yield a dried composite that comprises composite particles that are powder-like and/or composite structure(s) that are capable of being reduced to composite particles that are powder-like, wherein the composite particles comprise the enzyme component within a nanoporous organopolysiloxane matrix;
grinding, classifying, or grinding and classifying the composite structure(s) and/or composite particles to adjust one or more particle size parameters of the composite particles;
washing the dried composite, the composite structures, and/or the composite particles to reduce the amount of any unincorporated or exposed enzyme component therefrom; and
applying an enteric coating to the composite particle.

28. (canceled)

29. (canceled)

30. (canceled)

31. A stabilized enzyme product comprising composite particles that comprise an enzyme component within a nanoporous organopolysiloxane matrix wherein:
the enzyme component is capable of hydrolyzing one or more acyl-homoserine lactones;
the enzyme component constitutes about 0.01 wt % to about 40 wt % of the composite particles on a dry weight basis;
the composite particles have a density in a range of about 0.02 g/cm³ to about 0.5 g/cm³, an accessible surface area in a range of about 250 m²/g to about 600 m²/g, and fractal three-dimensional structures; and
the nanoporous organopolysiloxane is derived from a siloxane monomer component that comprises:
one or more tetraalkoxysilane monomers having the general formula Si(OR)₄ or (OR)₃Si(OSi(OR)₂)ₙOSi(OR)₃; and
one or more organo-alkoxysiloxane monomers having the general formula Si(OR)₄₋ₓ₁R′ₓ;
wherein:
n is in the range of 0 to 100;
x is in the range of 1 to 3;
each of the R groups is selected from the group consisting of methyl, ethyl, propyl, isopropyl, and butyl alkyl; and
each R′ is selected from the group consisting of methyl, ethyl, propyl, isopropyl, butyl, octyl, hexadecyl, octadecyl, phenyl, trimethylaminopropyl, aminopropyl, aminobutyl, methylphosphonyl, hydroxymethal, acetoxymethyl, acetoxypropyl, acetoxypolyethyleneoxypropyl, benzyl, carboxymethoxyethyl, expoxyhexyl, glycidoxypropyl, isocyanatopropyl, and phenethyl; and
wherein the one or more organo-alkoxysiloxane monomers are at an amount that is greater than 0 mol % and no more than about 30 mol % of the siloxane monomer component on a silicon mol basis.

32. The stabilized enzyme product of claim 31, wherein:
for the one or more tetraalkoxysilane monomers, R is methyl, ethyl, or propyl; and
for the one or more organo-alkoxysiloxane monomers, x is 2 or 3, R is methyl or ethyl, and R′ is methyl or ethyl.

33. The stabilized enzyme product of claim 31, wherein the amount of the organo-alkoxysiloxanes in the siloxane monomer component is in a range of about 5 mol % to and about 20 mol % on a silicon mol basis.

34-46. (canceled)

47. The stabilized enzyme product of claim 31, wherein the enzyme component comprises SEQ ID NO: 1, SEQ ID NO: 3, SEQ ID NO: 5, or SEQ ID NO: 7.

48. (canceled)

49. (canceled)

50. (canceled)

51. A method of hydrolyzing one or more acyl-homoserine lactones produced by one or more organisms of a gut microbiome within a gut of an individual, the method comprising administering an effective amount of a stabilized enzyme product to the patient such that the stabilized enzyme product reaches the gut of the patient, and, while in the gut, the enzyme component remains active within the nanoporous organopolysiloxane matrix and hydrolyzes the one or more acyl-homoserine lactones produced by one or more organisms of a gut microbiome within the gut of the patient, wherein the stabilized enzyme product comprises composite particles that comprise an enzyme component within a nanoporous organopolysiloxane matrix wherein:
the enzyme component is capable of hydrolyzing one or more acyl-homoserine lactones;
the enzyme component constitutes about 0.01 wt % to about 40 wt % of the composite particles on a dry weight basis;
the composite particles have a density in a range of about 0.02 q/cm³ to about 0.5 g/cm³, an accessible surface area in a range of about 250 m²/g to about 600 m²/g, and fractal three-dimensional structures; and
the nanoporous organopolysiloxane is derived from a siloxane monomer component that comprises:
one or more tetraalkoxysilane monomers having the general formula Si(OR)₄ or (OR)₃Si(OSi(OR)₂)ₙOSi(OR)₃; and
one or more organo-alkoxysiloxane monomers having the general formula Si(OR)₄₋ₓR′ₓ;
wherein:
n is in the range of 0 to 100;
x is in the range of 1 to 3;
each of the R groups is selected from the group consisting of methyl, ethyl, propyl, isopropyl, and butyl alkyl; and
each R′ is selected from the group consisting of methyl, ethyl, propyl, isopropyl, butyl, octyl, hexadecyl, octadecyl, phenyl, trimethylaminopropyl, aminopropyl, aminobutyl, methylphosphonyl, hydroxymethal, acetoxymethyl, acetoxypropyl, acetoxypolyethyleneoxypropyl, benzyl, carboxymethoxyethyl, expoxyhexyl, glycidoxypropyl, isocyanatopropyl, and phenethyl; and
wherein the one or more organo-alkoxysiloxane monomers are at an amount that is greater than 0 mol % and no more than about 30 mol % of the siloxane monomer component on a silicon mol basis.

52. The method of claim 51, wherein the hydrolyzing of the one or more acyl-homoserine lactones prevents or suppresses Proteobacteria overgrowth within the gut microbiome of the individual.

53. The method of claim 51, wherein the hydrolyzing of the one or more acyl-homoserine lactones prevents the individual from exhibiting, or reduces the degree to which the individual exhibits, one or more symptoms or disease indices associated with a disease or disorder of the individual's gut.

54. The method of claim 51, wherein the hydrolyzing of the one or more acyl-homoserine lactones maintains a relative abundance of bacteriodes and Firmicutes in the gut microbiome of the individual in a range that is normal for said individual.

55. The method of claim 54, wherein the individual is a human having a normal range of relative abundance of bacteroides in the gut microbiome of about 35% to about 85% and a normal range of relative abundance of Firmicutes in the gut microbiome of about 35% to about 85%.

56. (canceled)
